(19) United States
(12) Patent Application Publication
(10) Pub. No.: US 2012/0094902 A1
(43) Pub. Date: Apr. 19, 2012

(54) Fast-acting insulin formulation

(75) Inventors: Olivier Soula, Meyzieu (FR); Rémi Soula, Meyzieu (FR); Gerard Soula, Meyzieu (FR)

(73) Assignee: ADOCIA, Lyon (FR)

(21) Appl. No.: 13/287,793

(22) Filed: Nov. 2, 2011

Related U.S. Application Data
(63) Continuation-in-part of application No. 12/662,036, filed on Mar. 29, 2010.
(60) Provisional application No. 61/202,692, filed on Mar. 27, 2009.

(30) Foreign Application Priority Data
Mar. 27, 2009 (FR) 09 01478

(51) Int. Cl. A61K 38/28; C07K 1/107; A61P 3/10; C07K 14/62

(52) U.S. Cl. 514/5.9; 530/303

(57) ABSTRACT

A complex between an insulin and a polysaccharide comprising carboxyl functional groups is described, the polysaccharide being chosen from polysaccharides functionalized with at least one phenylalanine derivative, noted Phe, the phenylalanine derivative being chosen from phenylalanine and its alkali metal cation salts, phenylalaninol, phenylalaninamide and ethylbenzylamine or from phenylalanine esters, and the insulin being either a human insulin or an insulin analog. A pharmaceutical composition including at least one complex, especially in the form of an injectable solution, is also described.

The present application is a continuation-in-part application of U.S. patent application Ser. No. 12/662,036 filed Mar. 29, 2010, which claims priority to U.S. Provisional Application No. 61/202,692 filed Mar. 27, 2009 and to French application Ser. No. 09/01478 filed in France on Mar. 27, 2009. The disclosures of the prior applications are incorporated herein by reference in their entireties.

### BACKGROUND

The present invention relates to a fast-acting formulation of recombinant insulins, human or analogs.

Since the production of insulin by genetic engineering, at the start of the 1980s, diabetic patients have benefited from human insulin for their treatment. This product has greatly improved this therapy since the immunological risks associated with the use of non-human insulin, in particular porcine insulin, are eliminated.

One of the problems to be solved for improving the health of diabetic patients is to provide them with insulin formulations that provide a hypoglycemic response similar in terms of kinetics to the physiological response generated by the start of a meal, to prevent them from anticipating the start of their meal time and to perform an insulin injection at the start of the meal.

It is nowadays accepted that the provision of such formulations is essential for the best possible management of the disease.

Human insulin as formulated in its commercial form does not make it possible to obtain a hypoglycemic response that is close in terms of kinetics to the physiological response generated by the start of a meal in a healthy person, since, at the concentration of use (100 IU/mL), in the presence of zinc and other excipients such as phenol or cresol, it assembles in the form of a hexamer, whereas it is active in monomer and dimer form. Human insulin is in the form of hexamers and is stable for up to 2 years at 4° C. In the form of monomers, it has a very high propensity to aggregate and then to fibrilate, leading to a loss of activity.

Dissociation of the hexamers into dimers and of the dimers into monomers delays its action by nearly 30 minutes when compared with a rapid insulin analog (Brange J., et al., Advanced Drug Delivery Review, 35, 1999, 307-335).

Genetic engineering has provided a response with the development of rapid insulin analogs. These insulins are modified on one or two amino acids so as to be more rapidly distributed in the blood compartment after subcutaneous injection. These insulins, Lispro (Eli Lilly), Aspart (Novo Nordisk) and Glulisine (Sanofi) are stable insulin solutions generating a hypoglycemic response closer than regular human insulin to the physiological response generated by the start of a meal. Consequently, patients treated with these rapid insulin analogs no longer have to anticipate their meal time, but can perform the insulin injection at the start of the meal.

The principle of rapid insulin analogs is to form hexamers at a concentration of 100 IU/mL to ensure the stability of the insulin in the commercial product, while at the same time promoting very fast dissociation of these hexamers into monomers after injection so as to obtain a rapid action.

Therefore, insulin analogs represent an improvement compared to regular human insulin in terms of kinetics of post-prandial glycemic reduction. However, there is still a need for an insulin formulation that has an even shorter action time than the one of insulin analogs so as to approach the kinetics of healthy patients.

The company Biodel proposed a solution to this problem, with a human insulin and insulin analogs formulation comprising EDTA and citric acid, as described in patent application US 2008/39365. EDTA, via its capacity to complex zinc atoms, and citric acid, via its interactions with the cationic parts, are described as destabilizing the hexameric form of insulins and thus reducing its action time.

However, such a formulation has several drawbacks.

Firstly, the injection of a solution containing citric acid may cause pain at the site of injection, which was indeed reported during various clinical studies performed by Biodel, Business Wire (Sep. 8, 2008).

Moreover, the use of a chelating agent such as EDTA, which is not specific for the zinc atom, may lead to side effects.

Since the use of fast-acting insulin is performed three times a day for type I and type II diabetics, the pain associated with the administration of the product is unacceptable to the patients, and the risks of possible side effects due to the excipients must be avoided by any mean.

There is thus a real and unsatisfied need for formulations that can significantly reduce the onset of action of injected insulin, either human or analog.

### SUMMARY

The present invention makes it possible to solve the various problems outlined above, by producing an insulin, either human or analog, formulation able to accelerate, after administration, the passage of human insulin or insulin analogs into the blood and/or to reduce faster glycemia compared to its corresponding commercial insulins products.

The present invention also makes it possible to reduce the onset of action of a fast-acting insulin analog formulation.

The invention consists in forming a complex of insulin with a polysaccharide comprising partially substituted carboxyl functional groups.

The formation of this complex may furthermore be performed by simple mixing of an aqueous insulin solution and an aqueous polysaccharide solution.

The invention also relates to the complex between an insulin and a polysaccharide comprising partially substituted carboxyl functional groups.

### BRIEF DESCRIPTION OF THE DRAWINGS

FIG. 1 represents the pharmacodynamics results obtained with the human insulin formulation described in Example 9, i.e., the concentration of Dglucose (in mM) as a function of the time post injection (in minutes). The curves show that the formulation comprising polysaccharide 1 and human insulin according to the invention (curve plotted with the squares corresponding to Example 9) makes it possible to obtain an onset of action of less than that of a commercial human insulin formulation (curve plotted with the triangles corresponding to Example 3).

FIG. 2 represents the pharmacodynamics results obtained with the human insulin formulation described in Example 10, i.e., the concentration of Dglucose (in mM) as a function of the time post injection (in minutes). The curves show that the formulation comprising polysaccharide 3 and human insulin according to the invention (curve plotted with the squares corresponding to Example 10) make it possible to obtain an onset of action lower than that of a commercial human insulin formulation (curve plotted with the triangles corresponding to Example 3).

FIG. 3 represents the pharmacodynamics results obtained with the human insulin formulation described in Example 11, i.e., the concentration of Dglucose (in mM) as a function of the time post injection (in minutes). The curves show that the formulation comprising polysaccharide 3 and an insulin analog according to the invention (curve plotted with the squares corresponding to Example 11) make it possible to obtain an onset of action lower than that of a commercial formulation of this insulin analog (curve plotted with the triangles corresponding to Example 1).

FIG. 4 represents the pharmacodynamics results obtained with the human insulin formulation described in Example 12, i.e., the concentration of Dglucose (in mM) as a function of the time post injection (in minutes). The curves show that the formulation comprising polysaccharide 1 and the human insulin according to the invention (curve plotted with the squares corresponding to Example 12) make it possible to obtain an onset of action lower than that of a commercial human insulin formulation (curve plotted with the triangles corresponding to Example 3).

FIG. 5 represents the results obtained by the Hummel Dreyer method and the Positive Peak Area (in mAu·s) as a function of Polymer Concentration (in mg/ml). The curve plotted with the squares corresponds to polysaccharide 1, the curve plotted with the triangles corresponds to polysaccharide 7 and the curve plotted with the circles corresponds to polysaccharide 6.

FIG. 6 represents the pharmacodynamics results obtained with the human insulin formulation described in Example 16, i.e., the concentration of Dglucose (in mM) as a function of the time post injection (in minutes). The formulation comprising polysaccharide 5 and human insulin according to the invention (curve plotted with the squares corresponding to Example 16, Tmin glucose=41±17 min) leads to a faster onset of action than that of a commercial human insulin formulation (curve plotted with the triangles corresponding to Example 3, Tmin glucose=61±31 min).

FIG. 7 represents the pharmacodynamics results obtained with the human insulin formulation described in Example 17, i.e., the concentration of Dglucose (in mM) as a function of the time post injection (in minutes). The formulation comprising polysaccharide 6 and human insulin according to the invention (curve plotted with the squares corresponding to Example 17, Tmin glucose=36±10 min) leads to a faster onset of action than that of a commercial human insulin formulation (curve plotted with the triangles corresponding to Example 3, Tmin glucose=61±31 min).

FIG. 8 represents the pharmacokinetics results obtained with the polysaccharide 6 human insulin formulation described in Example 17, i.e., the concentration of DInsulin (in mM) as a function of the time post injection (in minutes). The formulation comprising polysaccharide 6 and human insulin according to the invention (curve plotted with the squares corresponding to Example 17, Tmax insulin=18±9 min) leads to a faster onset of absorption than that of a commercial human insulin formulation (curve plotted with the triangles corresponding to Example 3, Tmax insulin=36±33 min).

FIG. 9 represents the pharmacodynamics results obtained with the human insulin formulation described in Example 21, i.e., the concentration of Dglucose (in mM) as a function of the time post injection (in minutes). The formulation comprising polysaccharide 6 and Humalog® according to the invention (curve plotted with the squares corresponding to Example 21, Tmin glucose=27±7 min) leads to a faster onset of action than that of a commercial Humalog® formulation (curve plotted with the triangles corresponding to Example 2, Tmin glucose=45±10 min).

FIG. 10 represents the pharmacokinetics results obtained with the polysaccharide 6 human insulin formulation described in Example 21, i.e. the concentration of DInsulin (in mM) as a function of the time post injection (in minutes). The formulation comprising polysaccharide 6 and Humalog® according to the invention (curve plotted with the squares corresponding to Example 21, Tmax insulin=11±5 min) leads to a faster onset of absorption than that of a commercial Humalog® formulation (curve plotted with the triangles corresponding to Example 2, Tmax insulin=28±13 min).

FIG. 11 represents the pharmacodynamics results obtained with the human insulin formulation described in Example 20, i.e., the concentration of Dglucose (in mM) as a function of the time post injection (in minutes). The formulation comprising polysaccharide 6 and NovoLog® according to the invention (curve plotted with the squares corresponding to Example 20, Tmin glucose=41±12 min) leads to a faster onset of action than that of a commercial NovoLog® formulation (curve plotted with the triangles corresponding to Example 1, Tmin glucose=57±15 min).

FIG. 12 represents the pharmacokinetics results obtained with the polysaccharide 6 human insulin formulation described in Example 20, i.e. the concentration of DInsulin (in mM) as a function of the time post injection (in minutes). The formulation comprising polysaccharide 6 and NovoLog® according to the invention (curve plotted with the squares corresponding to Example 20, Tmax insulin=12±7 min) leads to a faster onset of absorption than that of a commercial NovoLog® formulation (curve plotted with the triangles corresponding to Example 1, Tmax insulin=24±18 min).

### DETAILED DESCRIPTION

In one embodiment, the insulin is human insulin.

The term “human insulin” means an insulin obtained by synthesis or recombination, in which the peptide sequence is the sequence of human insulin, including the allelic variations and the homologs.

In one embodiment, the invention relates to the complex between human insulin and a polysaccharide comprising partially substituted carboxyl functional groups.

The invention also relates to the use of this complex for preparing human insulin formulations, which makes it possible, after administration, to accelerate the passage of insulin into the blood and/or to reduce faster glycemia compared to commercial human insulin products.

“Regular” human insulin formulations on the market at a concentration of 600 μM (100 IU/mL) have an onset of action of between 20 and 40 minutes and a glycemic nadir of between 60 and 120 minutes in the pig model and an onset of action of about 50-90 minutes and an offset of action of about 360-420 minutes in humans. The time to reach the maximum insulin concentration is between 90 and 120 minutes in humans.

The fast-acting insulin analog formulations on the market at a concentration of 600 μM (100 IU/mL) have an onset of action of between 15 and 30 minutes and a glycemic nadir of between 60 and 90 minutes in the pigs model and an onset of action of about 30-60 minutes and an offset of action of about 240-300 minutes in humans. The time to reach the maximum insulin concentration is between 50 and 80 minutes in humans.

The invention also relates to pharmaceutical compositions that comprises insulin and a polysaccharide comprising partially substituted carboxyl functional groups having the ability to form a complex with insulin.

The invention also relates to a method of preparing a human insulin formulation at an insulin concentration from 150 to 6000 μM (25 to 1000 IU/mL), the method utilizing a polysaccharide comprising partially substituted carboxyl functional groups having the ability to form a complex with insulin.

The invention also relates to a method of preparing a human insulin formulation at an insulin concentration in the region of 600 μmol/L (100 IU/mL), whose onset of action in human is less than 60 minutes, the method utilizing a polysaccharide comprising partially substituted carboxyl functional groups having the ability to form a complex with insulin.

The invention more particularly relates to the use of a complex according to the invention for the preparation of a “fast-acting” human insulin formulation.

The invention relates to the use of the complex according to the invention for preparing human insulin formulations at a concentration in the region of 600 μM (100 IU/mL), whose onset of action in human is less than 60 minutes, preferably less than 45 minutes and even more preferably less than 30 minutes.

The invention relates to the use of the complex according to the invention for preparing human insulin formulations at a concentration from 150 to 6000 μM (25 to 1000 IU/mL).

The invention relates to the use of the complex according to the invention for preparing human insulin formulations at a concentration from 240 to 3000 μM (40 to 500 IU/mL).

The invention relates to the use of the complex according to the invention for preparing human insulin formulations at a concentration from 600 to 1200 μM (100 to 200 IU/mL).

In one embodiment, the insulin is an insulin analog. The term “insulin analog” means a recombinant insulin whose primary sequence contains at least one modification relative to the primary sequence of human insulin.

In one embodiment, the insulin analog is chosen from the group consisting of insulin Lispro (Humalog®), insulin Aspart (NovoLog®, Novorapid®) and insulin glulisine (Apidra®).

In one embodiment, the invention relates to the complex between an insulin analog and a polysaccharide comprising carboxyl functional groups.

In one embodiment, the invention relates to the complex between an insulin analog chosen from the group consisting of insulin Lispro (Humalog®), insulin Aspart (NovoLog®, Novorapid®) and insulin glulisine (Apidra®) and a polysaccharide comprising carboxyl functional groups.

The invention also relates to the use of this complex for preparing insulin analog formulations that make it possible to reach more quickly, after administration, a plasmatic level of insulin and/or a reduction of glucose than commercial insulin analog formulations.

The invention relates to the use of the complex according to the invention for preparing insulin analog formulations at a concentration in the region of 600 μM (100 IU/mL), whose onset of action in human is less than 30 minutes and preferably less than 20 minutes.

The invention also relates to a method of preparing an insulin analog formulation at an insulin concentration from 150 to 6000 μM (25 to 1000 IU/mL), the method utilizing a polysaccharide comprising partially substituted carboxyl functional groups having the ability to form a complex with insulin.

The invention also relates to a method of preparing an insulin analog formulation at an insulin concentration in the region of 600 μM (100 IU/mL), whose onset of action in human is less than 30 minutes, the method utilizing a polysaccharide comprising partially substituted carboxyl functional groups having the ability to form a complex with insulin.

In one embodiment, the invention relates to the use of the complex according to the invention for preparing insulin analogs formulations at a concentration from 150 to 6000 μM (25 to 1000 IU/mL).

The invention relates to the use of the complex according to the invention for preparing insulin analogs formulations at a concentration from 240 to 3000 μM (40 to 500 IU/mL).

The invention relates to the use of the complex according to the invention for preparing insulin analogs formulations at a concentration from 600 to 1200 μM (100 to 200 IU/mL).

In one embodiment, the polysaccharide comprising carboyxl functional groups is chosen from functionalized polysaccharides predominantly consisting of glycoside bonds of (1,6) type and, in one embodiment, the polysaccharide predominantly consisting of glycoside bonds of (1,6) type is a functionalized dextran comprising carboxyl functional groups.

Said polysaccharides are functionalized with at least one phenylalanine derivative, noted Phe:
said phenylalanine derivative being grafted or bonded onto the polysaccharides by coupling with an acid function, said acid function being an acid function borne by a linker arm R bonded to the polysaccharide via a function F, said function F resulting from coupling between the linker arm R and an —OH function of the polysaccharide,
F being either an ester, a carbamate or an ether function,
R being a chain comprising between 1 and 15 carbons, which is optionally branched and/or unsaturated, comprising one or more heteroatoms, such as O, N and/or S, and having at least one carboxyl function,
Phe being a residue of a phenylalanine derivative, L or D, produced from coupling between the phenylalanine amine and at least one acid borne by the group R

According to the invention, the functionalized polysaccharides may correspond to the following general formula I:

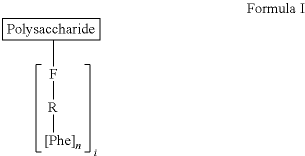

the polysaccharide being a dextran,
F resulting from coupling between the linker arm R and an —OH function of the polysaccharide and being either an ester, a carbamate or an ether function,
R being a chain comprising between 1 and 15 carbons, which is optionally branched and/or unsaturated, comprising one or more heteroatoms, such as O, N and/or S, and having at least one carboxyl function,
Phe being a residue of a phenylalanine derivative, L or D, produced from coupling between the amine of the phenylalanine derivative and at least one acid borne by the group R,

n represents the mole fraction of R substituted with Phe and is between 0.2 and 0.9, preferably between 0.3 and 0.8 and more preferably between 0.3 and 0.6,

i represents the average mole fraction of groups F-R-[Phe]ₙ borne per saccharide unit and is between 0.6 and 2.5, preferably between 0.8 and 2.2 preferably between 1.0 and 2.0;
when R is not substituted with Phe, then the acid(s) of the group R are carboxylates of a cation, preferably an alkali metal cation such as Na⁺ or K⁺.

The polysaccharide comprises on average at least 60 substituted or unsubstituted carboxylate units per 100 saccharide units.

In one embodiment, F is an ester function.

In one embodiment, F is a carbamate function.

In one embodiment, F is an ether function.

In one embodiment, the polysaccharide according to the invention is characterized in that the group R is chosen from the following groups:

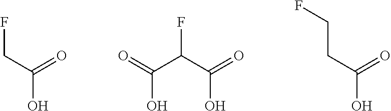

or the alkali metal cation salts thereof.

In one embodiment, the polysaccharide according to the invention is characterized in that F is an ether function and the group R is:

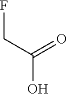

or the alkali metal cation salts thereof.

In one embodiment, the polysaccharide according to the invention is characterized in that F is a carbamate function and the group R is:

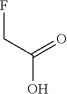

or the alkali metal cation salts thereof.

In one embodiment, the polysaccharide according to the invention is characterized in that the phenylalanine derivative is chosen from the group consisting of phenylalanine and alkali metal cation salts thereof, phenylalaninol, phenylalaninamide and ethylbenzylamine.

The polysaccharide may have a degree of polymerization of between 3 and 1000.

In one embodiment, it has a degree of polymerization of between 3 and 200.

In another embodiment, it has a degree of polymerization of between 3 and 50.

In one embodiment, the polysaccharide has a weight-average molecular weight of between 1 and 50 kg/mol and preferably between 5 and 10 kg/mol.

In one embodiment, the insulin is a human recombinant insulin as described in the European Pharmacopeia or US Pharmacopeia.

In one embodiment, the insulin is a human recombinant insulin chosen from the group consisting of Actrapid (Novo Nordisk), Humulin (Eli Lilly), Insuman (Sanofi), Wosulin (Wockhardt) or other biosimilar/generic versions such as the one from Biocon.

In one embodiment, the insulin is an insulin analog chosen from the group consisting of insulin Lispro (Humalog®), insulin Aspart (Novolog®, Novorapid®) and insulin glulisine (Apidra®) or other biosimilar/generic versions such as the ones from Biocon.

In one embodiment, the polysaccharide/insulin mass ratio are between 0.4 and 10.

In one embodiment, they are between 0.4 and 6.

In one embodiment, they are between 0.8 and 5.

In one embodiment, they are between 1.6 and 4.

In one embodiment, they are between 1.6 and 2.8.

Preferably, this composition is in the form of an injectable solution.

In one embodiment, the insulin concentration of the solutions is from 150 to 6000 μM (25 to 1000 IU/mL).

In one embodiment, the insulin concentration of the solutions is from 240 to 3000 μM (40 to 500 IU/mL).

In one embodiment, the insulin concentration of the solutions is from 600 to 1200 μM (100 to 200 IU/mL).

In one embodiment, the insulin concentration of the solutions is 600 μM, i.e. 100 IU/mL.

In one embodiment, the insulin concentration of 600 μM may be reduced by simple dilution, in particular for pediatric applications.

The invention also relates to a pharmaceutical composition according to the invention, characterized in that it is obtained by drying and/or lyophilization.

In the case of local and systemic releases, the envisioned administration modes are intravenous, subcutaneous, intradermal or intramuscular.

The formulation of the invention complies with traditional devices for insulin treatment like insulin syringes and pens.

The transdermal, oral, nasal, vaginal, ocular, buccal and pulmonary administration routes are also envisioned.

The invention also relates to the use of a complex according to the invention for the formulation of a solution of insulin, either human or analog, with a concentration of 100 IU/mL intended for implantable or transportable insulin pumps.

The main advantages of the invention are the increase in the % of patients under a value of HbA1c of 7%, the reduction in overall hypoglycemias, the reduction of the total insulin daily dose and the reduction of weight gain.

### Example 1

### Fast-Acting Insulin Aspart Solution at 100 IU/mL

This solution is a commercial solution of insulin Aspart sold by the company Novo Nordisk under the name Novolog® in the USA and Novorapid® in Europe. This product is a fast-acting insulin analog.

### Example 2

### Fast-Acting Insulin Lispro Solution at 100 IU/mL

This solution is a commercial solution of insulin Lispro sold by the company Eli Lilly under the name Humalog®. This product is a fast-acting insulin analog.

### Example 3

### Solution of Human Insulin at 100 IU/mL

This solution is a commercial solution from Novo Nordisk sold under the name Actrapid®. This product is a human insulin.

### Example 4

### Preparation of a Solution of Human Insulin at 200 IU/mL

60.4 g of water are added to 884.7 mg of human insulin comprising two Zn²⁺ per hexamer, and the pH is then adjusted from 5.7 to 3 by adding 8 mL of 0.1 N HCl. The solution is neutralized to pH 7.0 by adding 10 mL of 0.1 N NaOH. The concentration is then adjusted to 200 IU/mL with 43.08 mL of water. The final pH of this solution is 7.02. The solution is finally filtered through a 0.22 μm membrane.

### Example 5

### Preparation of a Solution of Human Insulin at 500 IU/mL

15 g of water are added to 0.5636 g of human insulin comprising two Zn²⁺ per hexamer, and the pH is then adjusted to acidic pH by adding 5.98 g of 0.1 N HCl. The solution is homogenized and then neutralized to pH 7.2 by adding 8.3 g of 0.1 N NaOH. The concentration is adjusted by addition of 0.76 g of water. The solution is homogenized and finally filtered through a 0.22 μm membrane.

### Example 6

### Preparation of the Excipients

Preparation of the 200 mM pH 7.0 Phosphate Buffer

A solution A of monosodium phosphate is prepared as follows: 1.2 g of NaH₂PO₄ (10 mmol) are solubilized in 50 mL of water in a graduated flask.

A solution B of disodium phosphate is prepared as follows: 1.42 g of Na₂HPO₄ (10 mmol) are solubilized in 50 mL of water in a graduated flask.

The 200 mM pH 7.0 phosphate buffer is obtained by mixing 3 mL of solution A with 7 mL of solution B.

Preparation of a 0.8 mM Tween 20 Solution

The Tween 20 solution is obtained by solubilizing 98 mg of Tween 20 (80 μmol) in 100 mL of water in a graduated flask.

Preparation of a 1.5 M Glycerol Solution

The glycerol solution is obtained by solubilizing 13.82 g of glycerol (150 mmol) in 100 mL of water in a graduated flask.

Preparation of a 130 mM M-Cresol Solution

The m-cresol solution is obtained by solubilizing 14.114 g of m-cresol (130 mmol) in 986.4 g of water in a graduated flask.

Preparation of a M-Cresol Glycerol Solution (96.6 mM M-Cresol and 566 mM Glycerine)

743 g of the 130 mM m-cresol solution are added to 52.1 g of glycerine at 1.5 M glycerol and then diluted by addition of 215 g of water. The m-cresol glycerine at 1.5 M glycerol solution is homogenized during 30 minutes and then filtered through a 0.22 μm membrane.

### Example 7

### Description of Polysaccharides

Polysaccharide 1 is a sodium dextran methylcarboxylate modified with the sodium salt of L-phenylalanine obtained from a dextran with a weight-average molecular weight of 10 kg/mol (Pharmacosmos, average degree of polymerization of 39) according to the process described in patent application FR 07/02316. The average mole fraction of sodium methylcarboxylates, optionally modified with L-phenylalanine, i.e. i in formula I, is 1.06. The average mole fraction of sodium methylcarboxylates modified with L-phenylalanine, i.e. n in formula I, is 0.43.

Polysaccharide 3 is a sodium dextran methylcarboxylate modified with the sodium salt of L-phenylalanine obtained from a dextran with a weight-average molecular weight of 10 kg/mol (Pharmacosmos, average degree of polymerization of 39) according to the process described in patent application FR 07/02316. The average mole fraction of sodium methylcarboxylates, optionally modified with L-phenylalanine, i.e. i in formula I, is 1.06. The average mole fraction of sodium methylcarboxylates modified with L-phenylalanine, i.e. n in formula I, is 0.5.

Polysaccharide 5 is a sodium dextran methylcarboxylate modified with the sodium salt of L-phenylalanine obtained from a dextran with a weight-average molecular weight of 10 kg/mol (Pharmacosmos, average degree of polymerization of 39) according to the process described in patent application FR 07/02316. The average mole fraction of sodium methylcarboxylates, optionally modified with L-phenylalanine, i.e. i in formula I, is 1.65. The average mole fraction of sodium methylcarboxylates modified with L-phenylalanine, i.e. n in formula I, is 0.39.

Polysaccharide 6 is a sodium dextran methylcarboxylate modified with the sodium salt of L-phenylalanine obtained from a dextran with a weight-average molecular weight of 5 kg/mol (Pharmacosmos, average degree of polymerization of 19) according to the process described in patent application FR 07/02316. The average mole fraction of sodium methylcarboxylates, optionally modified with L-phenylalanine, i.e. i in formula I, is 1.65. The average mole fraction of sodium methylcarboxylates modified with L-phenylalanine, i.e. n in formula I, is 0.39.

Polysaccharide 7 is a sodium dextran methylcarboxylate modified with the sodium salt of L-phenylalanine obtained from a dextran with a weight-average molecular weight of 5 kg/mol (Pharmacosmos, average degree of polymerization of 19) according to the process described in patent application FR 07/02316. The average mole fraction of sodium methylcarboxylates, optionally modified with L-phenylalanine, i.e. i in formula I, is 1.10. The average mole fraction of sodium methylcarboxylates modified with L-phenylalanine, i.e. n in formula I, is 0.41.

Polysaccharide 8 is a sodium dextran methylcarboxylate modified with the sodium salt of L-phenylalanine obtained from a dextran with a weight-average molecular weight of 5 kg/mol (Pharmacosmos, average degree of polymerization of 19) according to the process described in patent application FR 07/02316. The average mole fraction of sodium methylcarboxylates, optionally modified with L-phenylalanine, i.e. i in formula I, is 1.10. The average mole fraction of sodium methylcarboxylates modified with L-phenylalanine, i.e. n in formula I, is 0.59.

Polysaccharide 9 is a sodium dextran methylcarboxylate modified with the sodium salt of L-phenylalanine obtained from a dextran with a weight-average molecular weight of 5 kg/mol (Pharmacosmos, average degree of polymerization of 19) according to the process described in patent application FR 07/02316. The average mole fraction of sodium methylcarboxylates, optionally modified with L-phenylalanine, i.e. i in formula I, is 1.3. The average mole fraction of sodium methylcarboxylates modified with L-phenylalanine, i.e. n in formula I, is 0.59.

Polysaccharide 10 is a sodium dextran methylcarboxylate modified with the sodium salt of L-phenylalanine obtained from a dextran with a weight-average molecular weight of 5 kg/mol (Pharmacosmos, average degree of polymerization of 19) according to the process described in patent application FR 07/02316. The average mole fraction of sodium methylcarboxylates, optionally modified with L-phenylalanine, i.e. i in formula I, is 1.3. The average mole fraction of sodium methylcarboxylates modified with L-phenylalanine, i.e. n in formula I, is 0.35.

Polysaccharide 11 is a sodium dextran methylcarboxylate modified with the sodium salt of L-phenylalanine obtained from a dextran with a weight-average molecular weight of 5 kg/mol (Pharmacosmos, average degree of polymerization of 19) according to the process described in patent application FR 07/02316. The average mole fraction of sodium methylcarboxylates, optionally modified with L-phenylalanine, i.e. i in formula I, is 2.0. The average mole fraction of sodium methylcarboxylates modified with L-phenylalanine, i.e. n in formula I, is 0.5.

Polysaccharide 12 is a sodium dextran methylcarboxylate modified with the sodium salt of L-phenylalanine obtained from a dextran with a weight-average molecular weight of 1 kg/mol (Pharmacosmos, average degree of polymerization of 4) according to the process described in patent application FR 07/02316. The average mole fraction of sodium methylcarboxylates, optionally modified with L-phenylalanine, i.e. i in formula I, is 1.72. The average mole fraction of sodium methylcarboxylates modified with L-phenylalanine, i.e. n in formula I, is 0.42.

Polysaccharide 13 is a sodium dextran methylcarboxylate modified with the sodium salt of L-phenylalanine obtained from a dextran with a weight-average molecular weight of 1 kg/mol (Pharmacosmos, average degree of polymerization of 4) according to the process described in patent application FR 07/02316. The average mole fraction of sodium methylcarboxylates, optionally modified with L-phenylalanine, i.e. i in formula I, is 2.0. The average mole fraction of sodium methylcarboxylates modified with L-phenylalanine, i.e. n in formula I, is 0.5.

Polysaccharide 14 is a sodium N-methylcarboxylate dextran urethane modified with the sodium salt of L-phenylalanine obtained from a dextran with a weight-average molecular weight of 5 kg/mol (Pharmacosmos, average degree of polymerization of 19) according to the process described in patent U.S. application Ser. No. 13/250803. The average mole fraction of sodium N-methylcarboxylates, optionally modified with L-phenylalanine, i.e. i in formula I, is 1.82. The average mole fraction of sodium N-methylcarboxylates modified with L-phenylalanine, i.e. n in formula I, is 0.35.

### Example 8

### Preparation of a Polysaccharide Solution

A common preparation for the various polysaccharides solutions is given here.

The solution of a polysaccharide is obtained by solubilizing 2.0 g of this polysaccharide (water content=10%) in 56.9 mL of water in a 50 mL tube (concentration of a polysaccharide of 31.6 mg/mL).

### Example 9

### Preparation of a Solution of Human Insulin at 100 IU/mL in the presence of polysaccharide 1 ([polysaccharide 1]/[insulin] mass ratio of 2.0)

For a final volume of 50 mL of formulation with a [polysaccharide 1]/[insulin] mass ratio of 2.0, the various reagents are mixed together in the amounts specified in the table below and in the following order:

| | | |
|---|---|---|
| Human insulin at 200 IU/mL | 25 | mL |
| Polysaccharide 1 at 99.2 mg/mL | 3.61 | mL |
| pH 7.0 1M phosphate buffer | 500 | μL |
| 0.78 mM Tween 20 | 516 | μL |
| 1.5M glycerol | 621 | μL |
| 130 mM m-cresol | 11.15 | mL |
| Water (volume for dilution − volume of | 8.55 | mL |
| sodium hydroxide) | | |

The final pH is 7.0±0.3.

This clear solution is filtered through a 0.22 μm membrane and is then placed at +4° C.

### Example 10

### Preparation of a Solution of Human Insulin at 100 IU/mL in the Presence of Polysaccharide 3 (Polysaccharide 3]/[Insulin] Mass Ratio of 2.0)

For a final volume of 50 mL of formulation with a [polysaccharide 3]/[insulin] mass ratio of 2.0, the various reagents are mixed together in the amounts specified in the table below and in the following order:

| | | |
|---|---|---|
| Human insulin at 500 IU/mL | 10 | mL |
| Polysaccharide 3 at 31.6 mg/mL | 11.9 | mL |
| pH 7.0 1M phosphate buffer | 500 | μL |
| 1.5M glycerol | 5.67 | mL |
| 130 mM m-cresol | 11.16 | mL |
| 1 mM Tween 20 | 0.4 | mL |
| Water (volume for dilution − volume of | 10.4 | mL |
| sodium hydroxide) | | |

The final pH is 7.0±0.3.

This clear solution is filtered through a 0.22 μm membrane and is then placed at +4° C.

### Example 11

### Preparation of a Solution of Insulin Analog (Insulin Aspart) at 100 IU/mL in the Presence of Polysaccharide 3 ([Polysaccharide 3]/[Insulin Analog] Mass Ratio of 2.0)

For a final volume of 10 mL of formulation with a [polysaccharide 3]/[insulin analog] mass ratio of 2.0, the various reagents are mixed together in the amounts specified in the table below and in the following order:

| | |
|---|---|
| Solution of the commercial product NovoLog ® | 10 mL |
| Lyophilized polysaccharide 3 | 73 mg |
| Tween 20 | 98 μg |

The final pH is 7.0±0.3.

This clear solution is filtered through a 0.22 μm membrane and is then placed at +4° C.

### Example 12

### Preparation of a Solution of Human Insulin at 100 IU/mL in the Presence of Polysaccharide 1 ([Polysaccharide 1]/[Insulin] Mass Ratio of 4.0)

For a final volume of 50 mL of formulation with a [polysaccharide 1]/[insulin] mass ratio of 4.0, the various reagents are mixed together in the amounts specified in the table below and in the following order:

| | |
|---|---|
| Human insulin at 200 IU/mL | 25 mL |
| Polysaccharide 1 at 99.2 mg/mL | 7.22 mL |
| pH 7.0 1M phosphate buffer | 500 μL |
| 0.78 mM Tween 20 | 516 μL |
| 1.5M glycerol | 621 μL |

The final pH is 7.0±0.3.

This clear solution is filtered through a 0.22 μm membrane and is then placed at +4° C.

### Example 13

### Preparation of a Solution of Human Insulin at 100 IU/mL in the Presence of Polysaccharide 3 (Polysaccharide 3]/[Insulin] Mass Ratio of 2.0)

A variant of the human insulin formulation with polysaccharide 3 described in Example 10 is prepared in the absence of phosphate. This solution otherwise has the same composition and a pH also of 7.0±0.3.

### Example 14

### Preparation of a Solution of Human Insulin at 100 IU/mL in the Presence of Polysaccharide 3 (Polysaccharide 3]/[Insulin] Mass Ratio of 2.0)

A variant of the human insulin formulation with polysaccharide 3 described in Example 10 is prepared in the absence of phosphate and of Tween. This solution otherwise has the same composition and a pH also of 7.0±0.3.

### Example 15

### Preparation of a Solution of Human Insulin at 100 IU/mL in the Presence of Polysaccharide 1 ([Polysaccharide 1]/[Insulin] Mass Ratio of 2.0)

For a final volume of 1300 mL of formulation with a [polysaccharide 1]/[insulin] mass ratio of 2.0, the various reagents are mixed together in the amounts specified in the table below and in the following order:

| | | |
|---|---|---|
| Human insulin at 500 IU/mL | 260 | mL |
| Polysaccharide 1 at 42.5 mg/mL | 223.3 | mL |
| 96.6 mM m-cresol/566 mM glycerol | 390 | mL |
| Water (volume for dilution − volume of | 426.7 | mL |
| sodium hydroxide) | | |

The final pH is 7.0±0.3.

This clear solution is filtered through a 0.22 μm membrane and is then placed at +4° C.

### Example 16

### Preparation of a Solution of Human Insulin at 100 IU/mL in the Presence of Polysaccharide 5 ([Polysaccharide 5]/[Insulin] Mass Ratio of 2.0)

For a final volume of 100 mL of formulation with a [polysaccharide 5]/[insulin] mass ratio of 2.0, the various reagents are mixed together in the amounts specified in the table below and in the following order:

| | |
|---|---|
| Human insulin at 500 IU/mL | 20 mL |
| Polysaccharide 5 at 34.7 mg/mL | 21.06 mL |
| 96.6 mM m-cresol/566 mM glycerol | 30 mL |
| Water (volume for dilution − volume of | 28.94 mL |
| sodium hydroxide) | |

The final pH is 7.0±0.3.

This clear solution is filtered through a 0.22 μm membrane and is then placed at +4° C.

### Example 17

### Preparation of a Solution of Human Insulin at 100 IU/mL in the Presence of Polysaccharide 6 ([Polysaccharide 6]/[Insulin] Mass Ratio of 2.0)

For a final volume of 100 mL of formulation with a [polysaccharide 6]/[insulin] mass ratio of 2.0, the various reagents are mixed together in the amounts specified in the table below and in the following order:

| | |
|---|---|
| Human insulin at 500 IU/mL | 20 mL |
| Polysaccharide 6 at 32.2 mg/mL | 22.62 mL |
| 96.6 mM m-cresol/566 mM glycerol | 30 mL |
| Water (volume for dilution − volume of | 27.38 mL |
| sodium hydroxide) | |

The final pH is 7.0±0.3.

This clear solution is filtered through a 0.22 μm membrane and is then placed at +4° C.

### Example 18

### Preparation of a Solution of Human Insulin at 100 IU/mL in the Presence of Polysaccharide 13 ([Polysaccharide 13]/[Insulin] Mass Ratio of 2.0)

For a final volume of 100 mL of formulation with a [polysaccharide 13]/[insulin] mass ratio of 2.0, the various reagents are mixed together in the amounts specified in the table below and in the following order:

| | |
|---|---|
| Human insulin at 500 IU/mL | 20 mL |
| Polysaccharide 13 at 27.71 mg/mL | 28.4 mL |
| 96.6 mM m-cresol/566 mM glycerol | 30 mL |
| Water (volume for dilution − volume of | 21.6 mL |
| sodium hydroxide) | |

The final pH is adjusted to 7.0±0.3.

This clear solution is filtered through a 0.22 μm membrane and is then placed at +4° C.

### Example 19

### Preparation of a Solution of Human Insulin at 500 IU/mL in the Presence of Polysaccharide 6 ([Polysaccharide 6]/[Insulin] Mass Ratio of 2.0)

For a final volume of 100 mL of formulation with a [polysaccharide 6]/[insulin] mass ratio of 2.0, the various reagents are mixed together in the amounts specified in the table below and in the following order:

| | |
|---|---|
| Human insulin at 500 IU/mL | 100 mL |
| Lyophilizate containing | |
| Polysaccharide 6 | 3650 mg |
| m-cresol | 2.9 mmol (0.31 g) |
| glycerol | 17 mmol (1.56 g) |

The solution is homogenized.

The final pH is adjusted to 7.0±0.3.

This clear solution is filtered through a 0.22 μm membrane and is then placed at +4° C.

### Example 20

### Preparation of a Solution of Insulin Analog (Insulin Aspart) at 100 IU/mL in the Presence of Polysaccharide 6 ([Polysaccharide 6]/[Insulin Analog] Mass Ratio of 2.0)

For a final volume of 100 mL of formulation with a [polysaccharide 6]/[insulin analog] mass ratio of 2.0, the various reagents are mixed together in the amounts specified in the table below and in the following order:

| | |
|---|---|
| Lyophilized polysaccharide 6 | 730 mg |
| Solution of the commercial product NovoLog ® | 100 mL |

The final pH is 7.0±0.3.

This clear solution is filtered through a 0.22 μm membrane and is then placed at +4° C.

### Example 21

### Preparation of a Solution of Insulin Analog (Insulin Lispro) at 100 IU/mL in the Presence of Polysaccharide 6 ([Polysaccharide 6]/[Insulin Analog] Mass Ratio of 2.0)

For a final volume of 100 mL of formulation with a [polysaccharide 6]/[insulin analog] mass ratio of 2.0, the various reagents are mixed together in the amounts specified in the table below and in the following order:

| | |
|---|---|
| Lyophilized polysaccharide 6 | 730 mg |
| Solution of the commercial product Humalog ® | 100 mL |

The final pH is 7.0±0.3.

This clear solution is filtered through a 0.22 μm membrane and is then placed at +4° C.

### Example 22

### Preparation of a Solution of Insulin Analog (Insulin Lispro) at 100 IU/mL in the Presence of Polysaccharide 7 ([Polysaccharide 7]/[Insulin Analog] Mass of 2.0)

For a final volume of 100 mL of formulation with a [polysaccharide 7]/[insulin analog] mass ratio of 2.0, the various reagents are mixed together in the amounts specified in the table below and in the following order:

| | |
|---|---|
| Lyophilized polysaccharide 7 | 730 mg |
| Solution of the commercial product Humalog ® | 100 mL |

The final pH is 7.0±0.3.

This clear solution is filtered through a 0.22 μm membrane and is then placed at +4° C.

### Example 23

### Preparation of a Solution of Insulin Analog (Insulin Lispro) at 100 IU/mL in the Presence of Polysaccharide 14 ([Polysaccharide 14]/[Insulin Analog] Mass of 2.0)

For a final volume of 100 mL of formulation with a [polysaccharide 14]/[insulin analog] mass ratio of 2.0, the various reagents are mixed together in the amounts specified in the table below and in the following order:

| | |
|---|---|
| Lyophilized polysaccharide 14 | 730 mg |
| Solution of the commercial product Humalog ® | 100 mL |

The final pH is 7.0±0.3.

This clear solution is filtered through a 0.22 μm membrane and is then placed at +4° C.

### Example 24

### Preparation of a Solution of Insulin Analog (Insulin Lispro) at 200 IU/mL

The commercial Humalog® (insulin lispro) formulation was concentrated using AMICON Ultra-15 centifugation tubes with a 3 kDa cut-off. The AMICON tubes were first rinsed with 12 mL of deionized water. 12 mL of the commercial formulation were centrifuged during 35 minutes at 4000 g at 20° C. The volume of the retentate was measured and the concentration estimated by the retentate volume. All the retentates were pooled and the global concentration estimated (>200 IU/mL).

The concentration of this concentrated lispro solution was adjusted to 200 IU/mL by addition of the 100 IU/mL commercial Humalog® formulation. The concentrated lispro formulation presents the same excipients concentrations (m-cresol, glycerine, phosphate) than the commercial 100 IU/mL formulation).

The final pH is identical to the commercial Humalog® formulation.

This clear solution is filtered through a 0.22 μm membrane and is then placed at +4° C.

### Example 25

### Preparation of a Solution of Insulin Analog (Insulin Lispro) at 200 IU/mL in the Presence of Polysaccharide 6 ([Polysaccharide 6]/[Insulin Analog] Mass Ratio of 2.0)

For the preparation of this formulation, the solution of insulin lispro at 200 UI/mL is prepared according to example 24.

For a final volume of 100 mL of formulation with a [polysaccharide 6]/[insulin analog] mass ratio of 2.0, the various reagents are mixed together in the amounts specified in the table below and in the following order:

| | |
|---|---|
| Lyophilized polysaccharide 6 | 1460 mg |
| Solution of insulin lispro at 200 UI/mL | 100 mL |

The final pH is adjusted to 7.0±0.3.

This clear solution is filtered through a 0.22 μm membrane and is then placed at +4° C.

### Example 26

### Human Insulin Solubilization at its Isolelectric Point

Human Insulin (rH insulin) has an isoelectric point at pH 5.3. The insulin precipitates at its isoelectric point. A test proving the formation of a complex between a polysaccharide and the insulin molecule is executed at the isoelectric point of insulin. If an interaction exists, it is possible to solubilize insulin at its isoelectric point.

A solution of human insulin at 200 IU/mL is prepared. Different solutions of polysaccharide at various concentrations in water are prepared. The polysaccharide solutions are added to the insulin solution (50/50 v/v mixture) to lead to 100 IU/mL insulin solutions with different polysaccharide concentrations. The pH of each solution is adjusted to pH 5.3 by addition of 200 mM acetic acid.

The aspect of the solution is documented. If the solution is turbid, the polysaccharide does not allow the solubilization of insulin at its isoelectric point. If the solution is clear, the polysaccharide does allow the solubilization of insulin at its isoelectric point.

By this way the minimum concentration of polysaccharide necessary to solubilize insulin at 100 IU/mL at its isoelectric point is determined. The lower the polysaccharide concentration needed, the higher the affinity of the polysaccharide for the insulin molecule.

| | Minimal concentration for |
|---|---|
| Polysaccharide | solubilization (mg/mL) |
| polysaccharide 1 | 1 |
| polysaccharide 5 | 0.9 |
| polysaccharide 7 | 3-3.7 |
| polysaccharide 6 | 1.2 |
| polysaccharide 8 | 1.5 |
| polysaccharide 9 | 0.7 |
| polysaccharide 10 | 2.5 |
| polysaccharide 11 | 0.7 |

### Example 27

### Insulin Analog Solubilization at its Isolelectric Point

Commercial fast acting insulin analogs have an isoelectric point around pH 5. The insulin analog precipitates near its isoelectric point. A test proving the formation of a complex between polysaccharides and the insulin analog molecule is executed at the isoelectric point of the insulin analog. If an interaction exists, it is possible to solubilize the insulin analog at its isoelectric point.

The commercial solution of insulin analog (NovoLog®, Apidra® or Humalog®) is dialysed against 1 mM PO4 (pH 7). After dialysis, the concentration of the analogs is at 90 IU/mL. The polysaccharide is weighted and solubilized by the dialyzed insulin analog solution to reach a polysaccharide/Insulin analog solution of 90 IU/mL of insulin analog and the desired polysaccharide concentration. The pH of each solution is adjusted to pH 5 by addition of a 200 mM acetic acid solution.

The aspect of the solution is documented. If the solution is turbid, the polysaccharide does not allow the solubilization of the insulin analog at its isoelectric point. If the solution is clear, the polysaccharide does allow the solubilization of the insulin analog at its isoelectric point.

By this way the minimum concentration of polysaccharide necessary to solubilize insulin analog at its isoelectric point is determined. The lower the polysaccharide concentration needed, the higher the affinity of the polysaccharide for the insulin analog molecule.

| | Minimal polysaccharide concentration for | | | |
|---|---|---|---|---|
| | solubilization (mg/mL) | | | |
| | rh- | Aspart | Lispro | Glulisine |
| Polysaccharide | Insulin | (NovoLog ®) | (Humalog ®) | (Apidra ®) |
| polysaccharide | 1 | Not tested | 1.6 | Not tested |
| 1 | | | | |
| polysaccharide | 3-3.7 | 2-4 | 3.5 | 2-4 |
| 7 | | | | |
| polysaccharide | 1.2 | 1-2 | 1.6 | 1-2 |
| 6 | | | | |

### Example 28

The polysaccharide/Insulin complex has been characterized by SEC-HPLC using the Hummel Dreyer method (Xianwen Lou, Qingshan Zhu, Ze Lei, Joost L. J. van Dongen, E. W. Meijer, Journal of Chromatography A, 1029 (2004) 67-75 and William R. Tschantz, Eric S. Furfine, and Patrick J. Casey, The Journal of Biological Chemistry, Vol. 272, No. 15, Issue of April 11, pp. 9989-9993, 1997).

For this analysis, a mobile phase containing insulin (4 UI/Ml in a phosphate buffer) is used and a constant volume of polysaccharide solutions at different concentrations (from 0 to 40 mg/mL) is injected in the system.

The complex formation is characterized by two phenomenons:
the observation of a positive peak corresponding to the polysaccharide Insulin complex
the observation of a negative peak eluted after the complex corresponding to the insulin depletion caused by the insulin consumed by the polysaccharide to form the complex.

The interaction between the insulin and the polysaccharide is measured by integration of the positive peak using an insulin specific detection (UV, I=276 nm).

The Hummel Dreyer method has been used to characterize polysaccharide Insulin complex with the polysaccharides 6, 7 and 1.

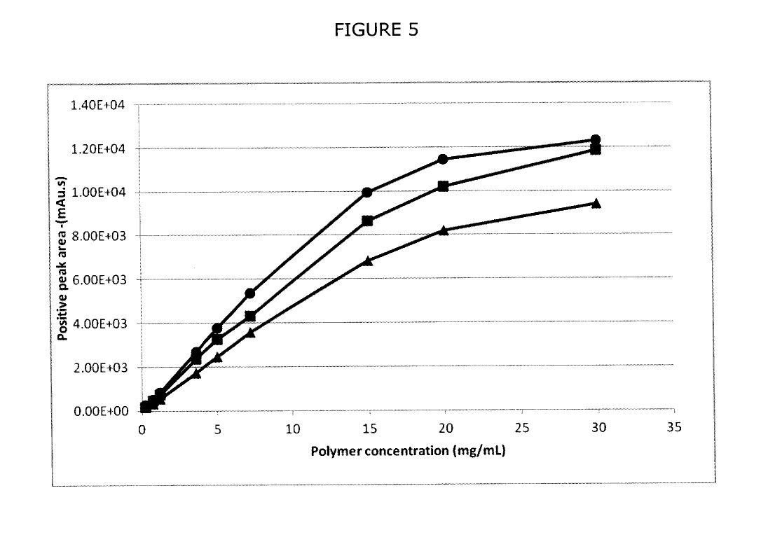

For the three different polysaccharides, the Hummel Dreyer method has allowed confirming the interaction between the insulin and the polysaccharides, see FIG. 5, on which curve plotted with the squares corresponding to polysaccharide 1, curve plotted with the triangles corresponding to polysaccharide 7 and curve plotted with the circles corresponding to polysaccharide 6.

### Example 29

### Injectability of the Solutions

All these solutions are injectable with the usual insulin injection systems. The formulations of the Polysaccharides with insulins described in Examples 9 to 25 are injected just as easily as the commercial products described in Examples 1 to 3 with insulin syringes with 31 gauges needles as with Novo Nordisk insulin pens, sold under the name Novopen®, equipped with 31 gauges needles.

### Example 30

### Protocol for Measuring the Pharmacodynamics of the Insulin Solutions

6 domestic pigs weighing about 50 kg, catheterized beforehand in the jugular vein, are fasted for 2 to 3 hours before the start of the experiment. In the hour preceding the injection of insulin, 3 blood samples are taken in order to determine the basal glucose level.

The injection of insulin at a dose of 0.125 IU/kg is performed subcutaneously into the neck, under the animal's ear using a Novopen insulin pen equipped with a 31 G needle.

Blood samples are then taken every 10 minutes over 3 hours and then every 30 minutes up to 5 hours. After taking each sample, the catheter is rinsed with a dilute heparin solution.

A drop of blood is taken to determine the glycemia using a glucometer.

The glucose pharmacodynamics curves are then plotted.

### Example 31

### Pharmacodynamics Results for the Insulin Solutions

| | | | Polysaccharide/ | Number of |
|---|---|---|---|---|
| Example | Insulin | Polysaccharide | insulin mass ratio | pigs |
| 1 | Aspart | — | — | 24 |
| 3 | Human | — | — | 31 |
| 9 | Human | 1 | 2.0 | 24 |
| 10 | Human | 3 | 2.0 | 9 |
| 11 | Aspart | 3 | 2.0 | 11 |
| 12 | Human | 1 | 4.0 | 5 |

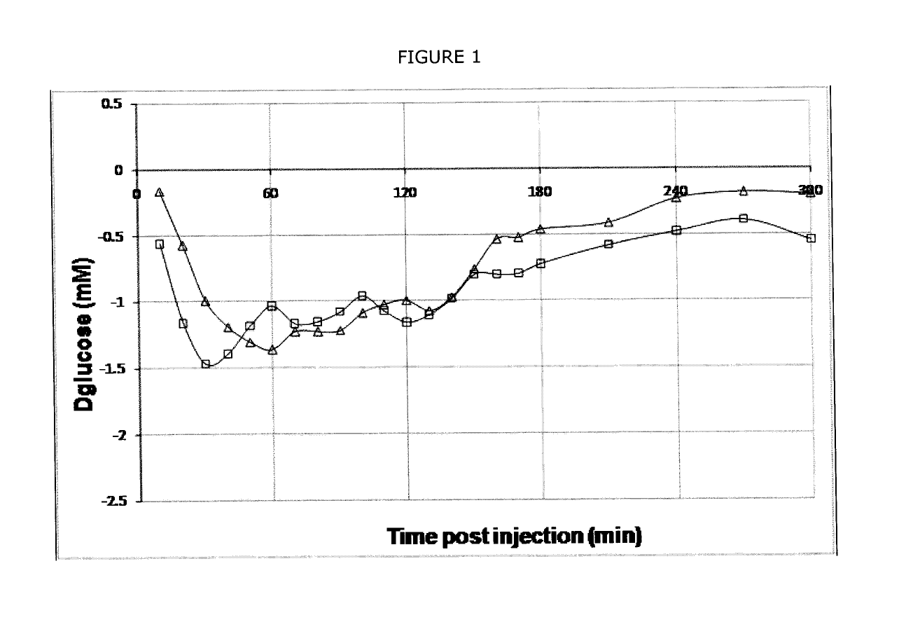

The results obtained with the human insulin formulation described in Example 9 are represented by the curves in FIG. 1. The curves show that the formulation comprising polysaccharide 1 and human insulin according to the invention (curve plotted with the squares corresponding to Example 9) makes it possible to obtain an onset of action of less than that of a commercial human insulin formulation (curve plotted with the triangles corresponding to Example 3).

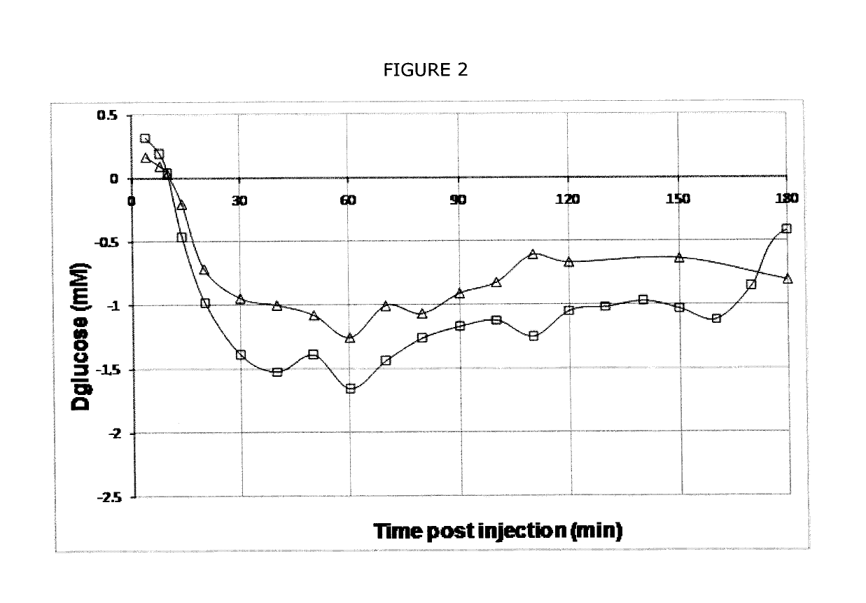

The results obtained with the human insulin formulation described in Example 10 are represented by the curves in FIG. 2. The curves show that the formulation comprising polysaccharide 3 and human insulin according to the invention (curve plotted with the squares corresponding to Example 10) make it possible to obtain an onset of action lower than that of a commercial human insulin formulation (curve plotted with the triangles corresponding to Example 3).

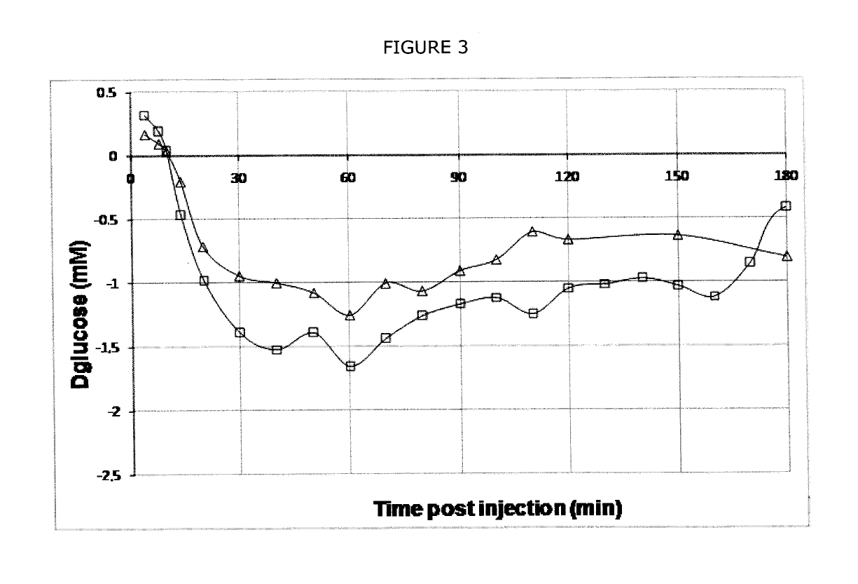

The results obtained with the insulin analog formulation described in Example 11 are represented by the curves in FIG. 3. The curves show that the formulation comprising polysaccharide 3 and an insulin analog according to the invention (curve plotted with the squares corresponding to Example 11) make it possible to obtain an onset of action lower than that of a commercial formulation of this insulin analog (curve plotted with the triangles corresponding to Example 1).

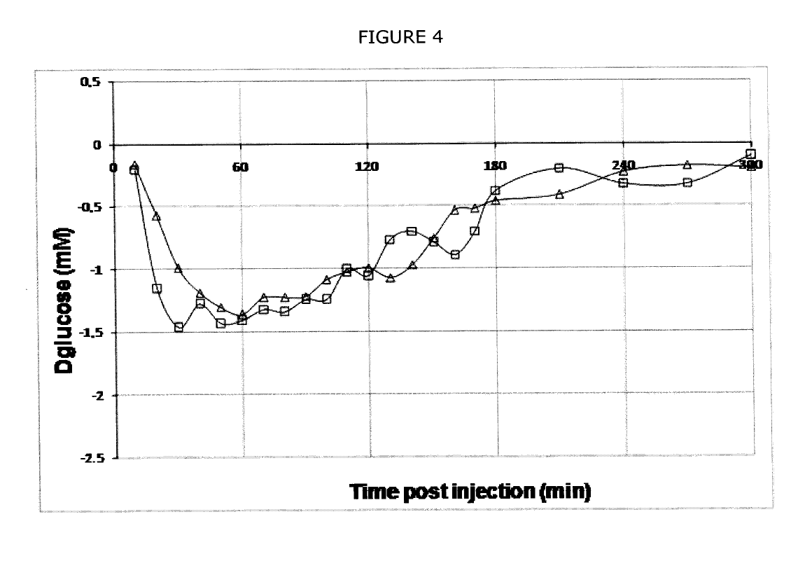

The results obtained with the human insulin formulation described in Example 12 are represented by the curves in FIG. 4. The curves show that the formulation comprising polysaccharide 1 and the human insulin according to the invention (curve plotted with the squares corresponding to Example 12) make it possible to obtain an onset of action lower than that of a commercial human insulin formulation (curve plotted with the triangles corresponding to Example 3).

### Example 32

### Second Protocol for Measuring the Pharmacodynamics of the Insulin Solutions

6 domestic pigs weighing about 50 kg, catheterized beforehand in the jugular vein, are fasted for 2 to 3 hours before the start of the experiment. In the hour preceding the injection of insulin, 3 blood samples are taken in order to determine the basal glucose level.

The injection of insulin at a dose of 0.125 IU/kg is performed subcutaneously into the neck, under the animal's ear using a Novopen insulin pen equipped with a 31 G needle.

Blood samples are taken every 4 minutes up to 20 minutes, then every 10 minutes up to 3 hours.

After taking each sample, the catheter is rinsed with a dilute heparin solution.

A drop of blood is taken to determine the glycemia using a glucometer.

Glucose pharmacodynamics curves are then plotted. The time for the individual minimal glucose levels are measured, averaged over the whole cohort and reported as Tmin glucose.

The remaining blood sample are collected on a dry tube then centrifuged to obtain serum.

Insulin levels of each pig sera is then measured in a sandwich Elisa assay.

Insulin pharmacokinetics curves are then plotted. The time for the individual maximal insulin levels are measured, averaged over the whole cohort and reported as Tmax insulin.

Comparison between formulations are only done for Pigs belonging to the same cohort.

### Example 33

| | | | Dose | Polysaccharide/ | Number |
|---|---|---|---|---|---|
| Example | Insulin | Polysaccharide | IU/kg | Insulin mass ratio | of pigs |
| 3 | Human | — | 0.125 | — | 11 |
| 16 | Human | 5 | 0.125 | 2.0 | 9 |

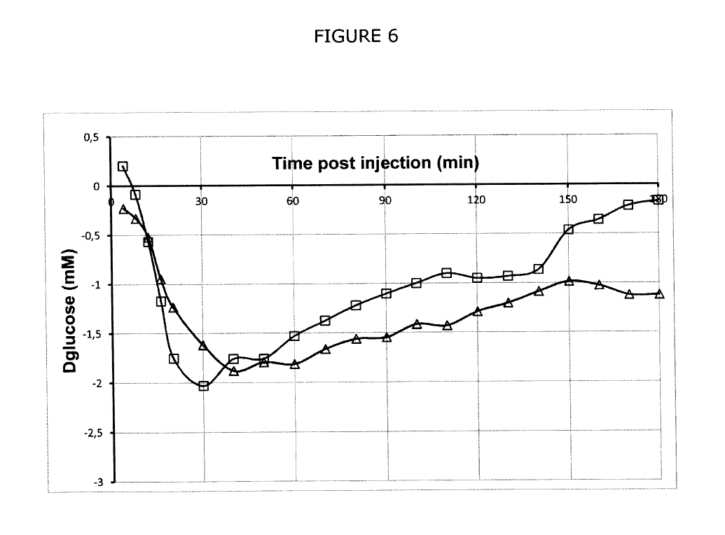

The pharmacodynamics results obtained with the human insulin formulation described in Example 16 are represented by the curves in FIG. 6. The formulation comprising polysaccharide 5 and human insulin according to the invention (curve plotted with the squares corresponding to Example 16, Tmin glucose=41±17 min) leads to a faster onset of action than that of a commercial human insulin formulation (curve plotted with the triangles corresponding to Example 3, Tmin glucose=61±31 min).

### Example 34

| | | Dose | | Polysaccharide/ | Nb of |
|---|---|---|---|---|---|
| Example | Insulin | IU/kg | Polysaccharide | insulin mass ratio | Pigs |
| 3 | Human | 0.125 | — | — | 11 |
| 17 | Human | 0.125 | 6 | 2.0 | 11 |

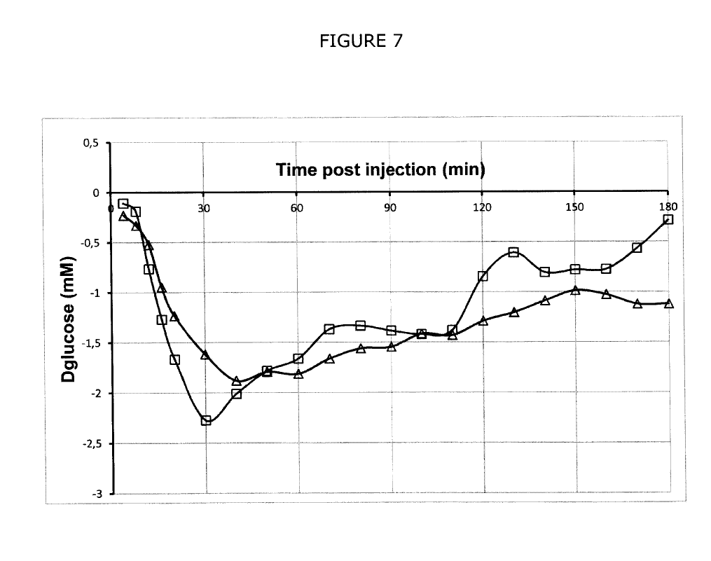

The pharmacodynamics results obtained with the human insulin formulation described in Example 17 are represented by the curves in FIG. 7. The formulation comprising polysaccharide 6 and human insulin according to the invention (curve plotted with the squares corresponding to Example 17, Tmin glucose=36±10 min) leads to a faster onset of action than that of a commercial human insulin formulation (curve plotted with the triangles corresponding to Example 3, Tmin glucose=61±31 min).

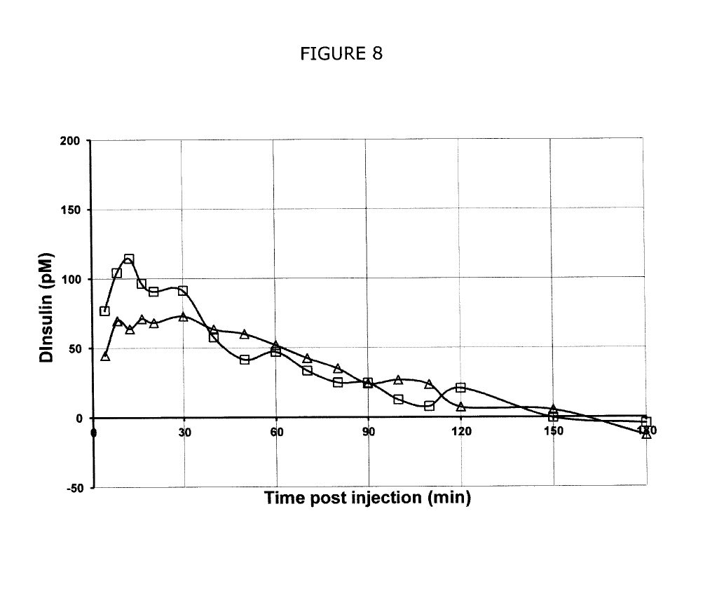

The pharmacokinetics results obtained with the polysaccharide 6 human insulin formulation described in Example 17 are represented by the curves in FIG. 8. The formulation comprising polysaccharide 6 and human insulin according to the invention (curve plotted with the squares corresponding to Example 17, Tmax insulin=18±9 min) leads to a faster onset of absorption than that of a commercial human insulin formulation (curve plotted with the triangles corresponding to Example 3, Tmax insulin=36±33 min).

### Example 35

| | | Dose | | Polysaccharide/ | Nb of |
|---|---|---|---|---|---|
| Example | Insulin | IU/kg | Polysaccharide | insulin mass ratio | Pigs |
| 2 | Lispro | 0.09 | — | — | 8 |
| 21 | Lispro | 0.09 | 6 | 2.0 | 8 |

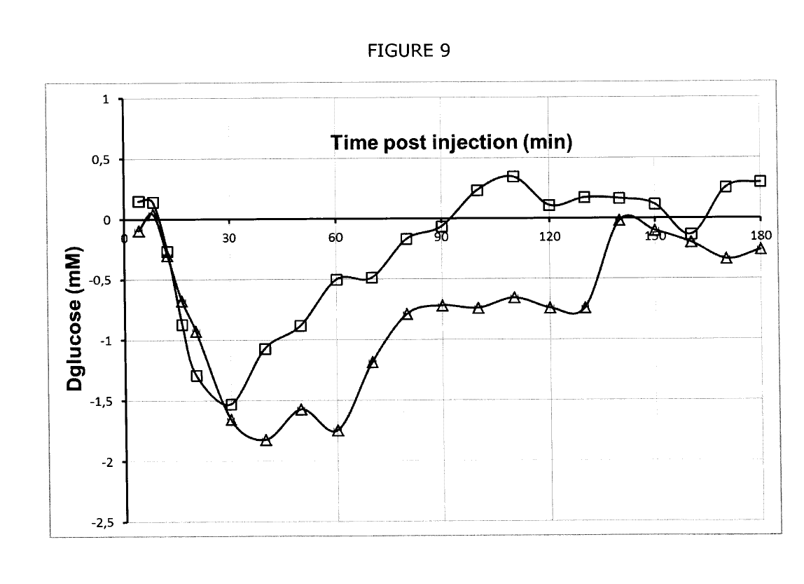

The pharmacodynamics results obtained with the analog formulation described in Example 21 are represented by the curves in FIG. 9. The formulation comprising polysaccharide 6 and Humalog® according to the invention (curve plotted with the squares corresponding to Example 21, Tmin glucose=27±7 min) leads to a faster onset of action than that of a commercial Humalog® formulation (curve plotted with the triangles corresponding to Example 2, Tmin glucose=45±10 min).

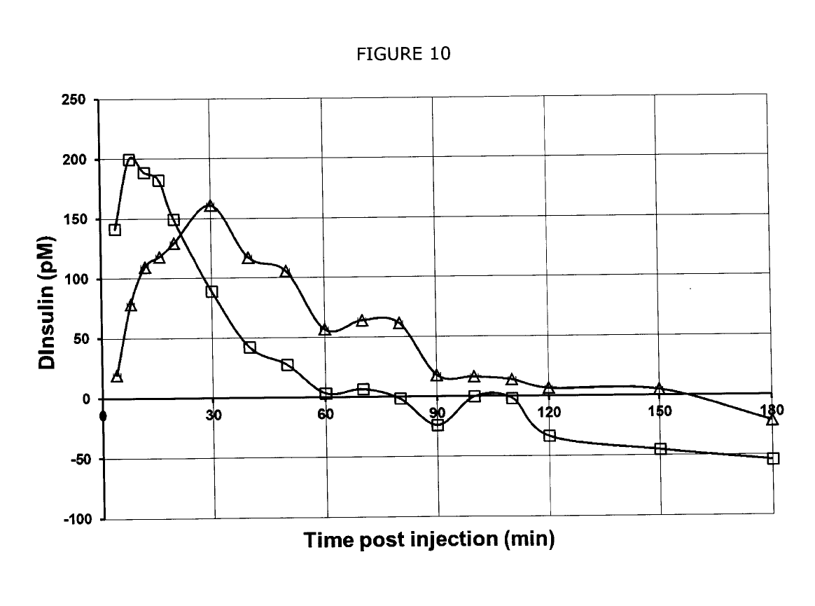

The pharmacokinetics results obtained with the polysaccharide 6 analog formulation described in Example 21 are represented by the curves in FIG. 10. The formulation comprising polysaccharide 6 and Humalog® according to the invention (curve plotted with the squares corresponding to Example 21, Tmax insulin=11±5 min) leads to a faster onset of absorption than that of a commercial Humalog® formulation (curve plotted with the triangles corresponding to Example 2, Tmax insulin=28±13 min).

### Example 36

| | | Dose | | Polysaccharide/ | Nb of |
|---|---|---|---|---|---|
| Example | Insulin | IU/kg | Polysaccharide | insulin mass ratio | Pigs |
| 2 | Lispro | 0.09 | — | — | 12 |
| 22 | Lispro | 0.09 | 7 | 2.0 | 11 |

According to the pharmacodynamics results of the formulation comprising polysaccharide 7 and Humalog® according to the invention (Tmin glucose=39±11 min) leads to a faster onset of action than that of a commercial Humalog® formulation (Tmin glucose=48±14 min).

According to the pharmacokinetics results of the formulation comprising polysaccharide 7 and Humalog® according to the invention (Tmax insulin=12±5 min) leads to a faster onset of absorption than that of a commercial Humalog® formulation (Tmax insulin=28±16 min).

### Example 37

| | | Dose | | Polysaccharide/ | Nb of |
|---|---|---|---|---|---|
| Example | Insulin | IU/kg | Polysaccharide | insulin mass ratio | Pigs |
| 1 | Aspart | 0.09 | — | — | 10 |
| 20 | Aspart | 0.09 | 6 | 2.0 | 10 |

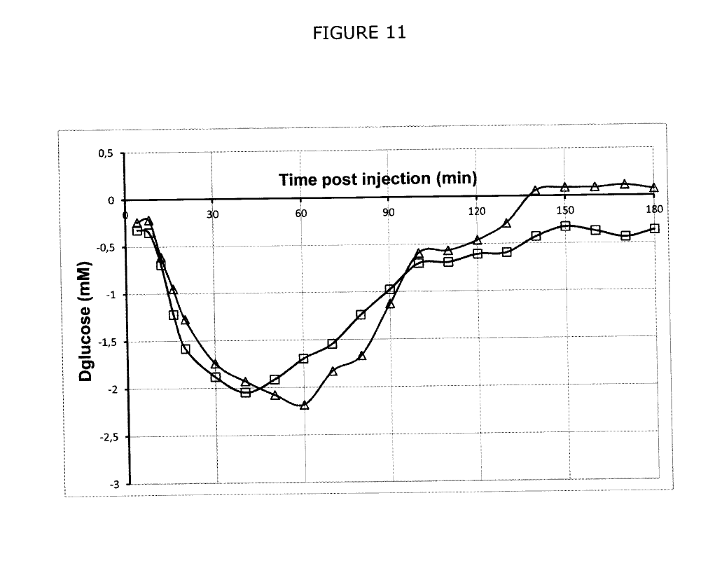

The pharmacodynamics results obtained with the analog formulation described in Example 20 are represented by the curves in FIG. 11. The formulation comprising polysaccharide 6 and NovoLog® according to the invention (curve plotted with the squares corresponding to Example 20, Tmin glucose=41±12 min) leads to a faster onset of action than that of a commercial NovoLog® formulation (curve plotted with the triangles corresponding to Example 1, Tmin glucose=57±15 min).

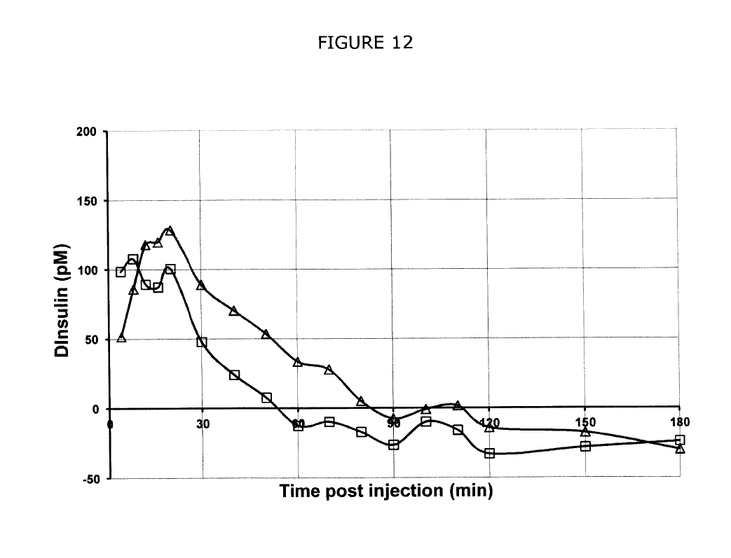

The pharmacokinetics results obtained with the polysaccharide 6 analog formulation described in Example 20 are represented by the curves in FIG. 12. The formulation comprising polysaccharide 6 and NovoLog® according to the invention (curve plotted with the squares corresponding to Example 20, Tmax insulin=12±7 min) leads to a faster onset of absorption than that of a commercial NovoLog® formulation (curve plotted with the triangles corresponding to Example 1, Tmax insulin=24±18 min).

### Example 38

| | | Dose | | Polysaccharide/ | Nb of |
|---|---|---|---|---|---|
| Example | Insulin | IU/kg | Polysaccharide | insulin mass ratio | Pigs |
| 3 | Human | 0.125 | — | — | 10 |
| 18 | Human | 0.125 | 13 | 2.0 | 9 |

According to the pharmacodynamics results of the formulation comprising polysaccharide 13 and human insulin according to the invention (Tmin glucose=46±20 min) leads to a faster onset of action than that of a commercial human insulin formulation (Tmin glucose=64±33 min).

According to the pharmacokinetics results of the formulation comprising polysaccharide 13 and human insulin according to the invention (Tmax insulin=12±6 min) leads to a faster onset of absorption than that of a commercial human insulin formulation (Tmax insulin=26±20 min).

### Example 39

| | | Dose | | Polysaccharide/ | Nb of |
|---|---|---|---|---|---|
| Example | Insulin | IU/kg | Polysaccharide | insulin mass ratio | Pigs |
| 24 | Lispro | 0.125 | — | — | 9 |
| 25 | Lispro | 0.125 | 6 | 2.0 | 9 |

According to the pharmacodynamics results of the formulation comprising polysaccharide 6 and insulin Lispro according to the invention (Tmin glucose=38±8 min) leads to a faster onset of action than that of the corresponding Lispro formulation (Tmin glucose=62±29 min).

According to the pharmacokinetics results of the formulation comprising polysaccharide 6 and insulin Lispro according to the invention (Tmax insulin=16±5 min) leads to a faster onset of absorption than that of the corresponding Lispro formulation (Tmax insulin=25±14 min).

### Example 40

| | | Dose | | Polysaccharide/ | Nb of |
|---|---|---|---|---|---|
| Example | Insulin | IU/kg | Polysaccharide | insulin mass ratio | Pigs |
| 2 | Lispro | 0.09 | — | — | 12 |
| 23 | Lispro | 0.09 | 14 | 2.0 | 11 |

According to the pharmacodynamics results of the formulation comprising polysaccharide 14 and Humalog® according to the invention (Tmin glucose=44±12 min) leads to a faster onset of action than that of a commercial Humalog® formulation (Tmin glucose=48±14 min).

According to the pharmacokinetics results of the formulation comprising polysaccharide 14 and Humalog® according to the invention (Tmax insulin=21±9 min) leads to a faster onset of absorption than that of a commercial Humalog® formulation (Tmax insulin=28±16 min).

### Example 41

### Phase I Clinical Trial

### Study Design

This phase I clinical trial was a single center, prospective, double blind randomized cross-over euglycemic clamp study. The study was a head-to-head comparison of polysaccharide 1/human insulin; (example 15) to fast-acting insulin analog, insulin aspart (example 1) and to regular human insulin (example 3). In 3 consecutive euglycemic clamp experiments, 12 healthy male caucasian volunteers (age: 27.2±6.5 years, BMI: 22.9±2.6 kg/m²) received 12 IU of the respective formulation and were monitored for 6 h.

Primary Study Objectives:

The primary objective was to assess the pharmacodynamic (PD) profile of example 15 after a single exposure, with the aim to demonstrate a faster onset of action in comparison to example 3. Glucose Infusion Rate (GIR) as determined by the euglycemic glucose clamp technique was the primary variable analysis.

Secondary Study Objectives:

The secondary objective was to confirm, by the comparison of the PD and the pharmacokinetics (PK) profiles, the superiority of example 15 to example 3 and the non-inferiority to example 1. The secondary variable was the serum insulin concentration profile.

Further secondary objectives were the safety and tolerability of example 15 after a single exposure. Adverse events, injection site reaction and laboratory safety parameters were the evaluated variables.

### Study Medications

Example 15: 100 IU/mL rhInsulin, 200 μM Zn²⁺, 29 mM m-cresol, 170 mM Glycerin, 7.3 mg/mL polysaccharide 1 (210-250 mOsm, pH 6.8±0.5)
Example 3: Actrapid® from Novo Nordisk, rhInsulin 100 IU/mL
Example 1: NovoLog® from Novo Nordisk, Insulin Aspart 100 IU/mL

### Primary Endpoint for Evaluation:

Time to reach maximal glucose-infusion-rate (T_{GIRmax}).

### Secondary Endpoints for Evaluation:

Time to half-maximal GIR during the initial rise (T_{GIR}_{_{—}}_{0.5max})
AUC for GIR between 0 and 180 minutes (AUC_{GIR}_{_{—}}₀₋₁₈₀ₘᵢₙ)
Time to reach INSmax (T_{INSmax})
Time to half-maximal insulin concentration in the serum during the initial rise (T_{INS}_{_{—}}_{0.5max})
AUC for INS between 0 and 60 minutes (AUC_{INS}_{_{—}}₀₋₆₀ₘᵢₙ)

### Safety Variables:

Adverse Events (AEs)
Safety laboratory
Injection site reactions

### Results on Efficacy:

Administration of example 15 results in a faster onset of action in comparison to example 3 as indicated by a trend for a shorter T_{GIRmax}, a strong trend for a faster T_{GIR}_{_{—}}_{0.5max} and a significantly lower AUC_{GIR}_{_{—}}₀₋₁₈₀ₘᵢₙ (Table 1). In addition, T_{INSmax} and T_{INS}_{_{—}}_{0.5} max are significantly shorter for example 15 than for example 3.

T_{INS}_{_{—}}_{0.5max} is significantly shorter for example 15 than for example 1 and T_{INSmax}, T_{GIR}_{_{—}}_{0.5max} and T_{GIRmax} are similar between the two medications.

### CONCLUSIONS

In humans, example 15 demonstrated a faster onset of absorption and a faster onset of action compared to example 3. Example 15 was also non inferior to example 1.

Example 15 is well tolerated in humans, without any indication of specific example 15 related difference in the safety profile compared to the two other insulins.

TABLE 1
Mean ± SD of primary and secondary efficacy variables of
the phase I clinical trial with polysaccharide Insulin formulation

| | Example 15 | Example 3 | Example 1 |
|---|---|---|---|
| T_{GIRmax} [min] | 92.9 ± 37.2 | 111.3 ± 42.2 | 91.3 ± 25.1 |
| T_{GIR}_{_{—}}_{0.5max} [min] | 59.9 ± 29.9 | 77.8 ± 23.9 | 55.4 ± 8.3 |
| AUC_{GIR}_{_{—}}₀₋₁₈₀ₘᵢₙ | 1262.8 ± 453.5 | 1001.3 ± 327.7 | 1393.4 ± 347.5 |
| [min*g/min/kg] | | | |
| T_{INSmax} [min] | 42.5 ± 33.0 | 67.5 ± 29.4 | 47.9 ± 12.3 |
| T_{INS}_{_{—}}_{0.5max} [min] | 13.1 ± 5.7 | 20.6 ± 6.6 | 25.1 ± 5.8 |
| AUC_{INS}_{_{—}}₀₋₆₀ₘᵢₙ | 13176 ± 2165 | 12193 ± 3485 | 20946 ± 8722 |
| [min*pmol/L] | | | |

1. A complex between an insulin and a polysaccharide comprising carboxyl functional groups, said polysaccharide being chosen from polysaccharides of formula I: 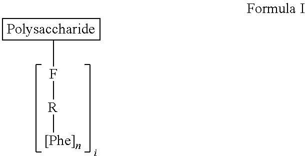
the polysaccharide being a dextran,
F resulting from coupling between the linker arm R and an —OH function of the polysaccharide and being either an ester, a carbamate or an ether function,
R being a chain comprising between 1 and 15 carbons, which is optionally branched and/or unsaturated, comprising one or more heteroatoms, and having at least one carboxyl function,
Phe being a residue of a phenylalanine derivative, L or D, produced from coupling between the amine of the phenylalanine derivative and at least one acid borne by the group R,
n represents the mole fraction of R substituted with Phe and is between 0.2 and 0.9,
i represents the average mole fraction of groups F-R-[Phe]ₙ borne per saccharide unit and is between 0.6 and 2.5;
when R is not substituted with Phe, then the acid(s) of the group R are carboxylates of a cation.

2. The complex as claimed in claim 1, wherein the polysaccharide has a degree of polymerization of between 3 and 1000.

3. The complex as claimed in claim 1, wherein the polysaccharide has a degree of polymerization of between 3 and 200.

4. The complex as claimed in claim 1, wherein the polysaccharide has a weight-average molecular weight of between 1 and 50 kg/mol.

5. The complex as claimed in claim 1, wherein the group R is chosen from the following groups: 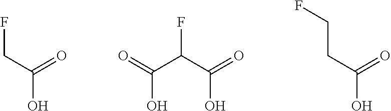
or the alkali metal cation salts thereof.

6. The complex as claimed in claim 1, wherein the phenylalanine derivative is chosen from the group consisting of phenylalanine and its alkali metal cation salts, phenylalaninol, phenylalaninamide and ethylbenzylamine.

7. The complex as claimed in claim 1, wherein the insulin is a human recombinant insulin.

8. The complex as claimed in claim 7, wherein the human recombinant insulin is chosen from the group consisting of Actrapid (Novo Nordisk), Humulin (Eli Lilly), Insuman (Sanofi), Wosulin (Wockhardt) and other biosimilar/generic versions.

9. The complex as claimed in claim 1, wherein the insulin is an insulin analog.

10. The complex as claimed in claim 9, wherein the insulin analog is chosen from the group consisting of insulin Lispro (Humalog®), insulin Aspart (Novolog®, Novorapid®) and insulin glulisine (Apidra®) and other biosimilar/generic versions.

11. The complex as claimed in 1, wherein the polysaccharide/insulin mass ratios are between 0.4 and 10.

12. The complex as claimed in 1, wherein the polysaccharide/insulin mass ratios are between 0.4 and 6.

13. The complex as claimed in 1, wherein the polysaccharide/insulin mass ratios are between 0.8 and 5.

14. A pharmaceutical composition comprising at least one complex according to claim 1.

15. A pharmaceutical composition comprising insulin and a polysaccharide comprising partially substituted carboxyl functional groups having the ability to form a complex according to claim 1.

16. A method of preparing a human insulin formulation at an insulin concentration from 150 to 6000 μM (25 to 1000 IU/mL), the method utilizing a polysaccharide comprising partially substituted carboxyl functional groups having the ability to form a complex according to claim 1.

17. A method of preparing a human insulin formulation at an insulin concentration in the region of 600 μmol/L (100 IU/mL), whose onset action in a human is less than 60 minutes, the method utilizing a polysaccharide comprising partially substituted carboxyl functional groups having the ability to form a complex according to claim 1.

18. A method of preparing an insulin analog formulation at an insulin concentration in the region of 600 μM (100 IU/mL), whose onset of action in a human is less than 45 minutes, the method utilizing a polysaccharide comprising partially substituted carboxyl functional groups having the ability to form a complex according to claim 1.

19. A method of preparing an insulin analog formulation at an insulin concentration in the region of 600 μM (100 IU/mL), whose onset of action in a human is less than 30 minutes, the method utilizing a polysaccharide comprising partially substituted carboxyl functional groups having the ability to form a complex according to claim 1.

20. The composition as claimed in claim 14, which is in the form of an injectable solution.

21. The composition as claimed in claim 20, wherein the insulin concentration of the solution is from 150 to 6000 μM (25 to 1000 IU/mL).

22. A method of preparing a human insulin formulation at an insulin concentration from 150 to 6000 μM (25 to 1000 IU/mL), the method utilizing the complex as claimed in claim 1.

23. A method of preparing a human insulin formulation at an insulin concentration in the region of 600 μmol/L (100 IU/mL), whose onset action in human is less than 60 minutes, the method utilizing the complex as claimed in claim 1.

24. A method of preparing an insulin analog formulation at an insulin concentration in the region of 600 μM (100 IU/mL), whose onset of action in human is less than 30 minutes, the method utilizing the complex as claimed in claim 1.

25. A method of preparing an insulin formulation at an insulin concentration of 100 IU/mL intended for injection pumps, the method utilizing the complex as claimed in claim 1.
